(19) United States
(12) Patent Application Publication
(10) Pub. No.: US 2026/0250762 A1
(43) Pub. Date: Aug. 27, 2026

(54) ASSAY OF RNA POLY(A) TAIL LENGTH

(71) Applicant: CellScript, LLC, Madison, WI (US)

(72) Inventors: Jerome J. Jendrisak, Middleton, WI (US); Ronald J. Meis, Brooklyn, WI (US); Kari Weber, Middleton, WI (US); Gary A. Dahl, Madison, WI (US)

(21) Appl. No.: 19/551,232

(22) Filed: Feb. 26, 2026

Related U.S. Application Data
(60) Provisional application No. 63/763,642, filed on Feb. 26, 2025.

(51) Int. Cl. C12Q 1/6876; C12Q 1/34; C12Q 1/6806; C40B 70/00

(52) U.S. Cl. CPC C12Q 1/6876; C12Q 1/34; C12Q 1/6806; C12Y 301/27005; C40B 70/00; C12Q 2600/166

(57) ABSTRACT

The present invention relates to compositions and kits and to methods, and uses of any thereof to accurately and precisely assay the size in terms of number of adenosines and the size distribution of polyadenylic acid (“polyA” or “poly(A)” tails) on the 3′-end portions of RNA molecules.

### FIELD OF THE INVENTION

The present invention relates to compositions and kits and to methods, and uses of any thereof to accurately and precisely assay the size in terms of number of adenosines and the size distribution of polyadenylic acid (“polyA” or “poly(A)” tails) on the 3′-end portions of RNA molecules.

### BACKGROUND

Eukaryotic mRNAs typically end with a sequence of adenosines at their 3′ end. These 3′ polyadenylic acid (“poly(A)” or “polyA” tails) can range in length from 20 to over 200 bases, depending on the organism, the specific mRNA, and other factors. PolyA tails play a critical role in mRNA metabolism, including stability, translation, nuclear export, and miRNA-mediated gene regulation.

In the field of mRNA therapeutics such as vaccines, gene editing, and gene or enzyme replacement therapies, manufacturing mRNA with consistent polyA tail length and size distribution is critical for consistent mRNA effectiveness.

Current compositions, kits, and methods known in the art for measuring the mean poly A tail length and the relative distribution of different polyA tail lengths released from an in vitro-synthesized or in vitro-manufactured mRNA molecule for prophylactic or therapeutic use include PCR-based approaches, next-generation sequencing, linked liquid chromatography and mass spectrometry (LC-MS) techniques, size exclusion chromatography (SEC) and ion-pair reverse-phase liquid chromatography (IP RP LC). These methods require specialized, expensive, and technically complex equipment that needs large amounts of time for maintenance, qualification, calibration, operation, and data interpretation by people having specialized knowledge and training. Some of these methods involve reverse transcription polymerase chain reaction (RT-PCR), which means that the results do not directly measure the polyA tail, but rather measure indirect products of a reverse transcriptase and DNA polymerase, or other nucleic acid modifying enzymes, as well as other of labeling steps or chemical reactions, all of which can result in inaccurate or biased polyA tail length results. Also, liquid chromatography methods like SEC and

IP RP HPLC use mixtures of organic chemicals, which generate chemical waste that must be collected, documented, stored and disposed of, at significant costs of time and money.

What is needed in the art are compositions, kits, kits-of-parts and methods and uses of any thereof to accurately, quickly, and easily assay the lengths of polyA tails and the distribution of poly A tail size of mRNAs that are synthesized or manufactured in vitro for use as products in basic, biological, medical, veterinary, and clinical research, gene editing, mRNA enzyme replacement therapies, base editing, genome editing, mRNA therapeutic, vaccine and other prophylactic applications in humans and animals, and for animal and plant breeding, and other agricultural applications. What is needed are compositions, kits, kits-of-parts and methods and uses of any thereof that are rapid and easy-to-use and easy to perform, and inexpensive, yet can be used to quantitate polyA tail lengths and tail length distributions for applications ranging from internal research, in-process testing of mRNAs that are being manufactured for use as therapeutic product, and for Quality Control assays, including by the QC Department for final pass-fail assay testing of final manufactured mRNA therapeutic products to assure that they satisfy all final product specifications. What is needed are compositions, kits, kits-of-parts and methods and uses of any thereof precisely measure the poly A tail length from an RNA sample from a natural source that comprises mRNA or other types of RNA, and for assaying internal stretches of polyA sequences within RNA molecules. What is needed are methods and uses that directly measure the lengths and distribution of lengths or polyA molecules rather than measuring such lengths by converting the poly A to cDNA using a reverse transcriptase or amplifying said cDNA made from polyA by PCR or another nucleic acid amplification method. What is needed are methods and uses that measure the lengths and distribution of lengths or poly A molecules by binding them to a surface or passing them through a column or resin or membrane, which can result in losses and errors. Preferably, what is needed are methods and uses that do not require large, equipment that is expensive to purchase, qualify, calibrate, maintain, and operate, and that do not require specialized knowledge and training to obtain and interpret the data or results of polyA lengths and distribution of lengths.

### BRIEF DESCRIPTION OF THE DRAWINGS

FIG. 1: Demonstration that staining gel with both berberine, in particular berberine hydrochloride, and SYBR Gold and produced more intense staining than either of these compounds did alone, enabling polyA tail lengths to be determined with only 5 picomoles of each RNase A-treated mRNA sample input and that the length of the 3′-poly(A)-tail added to an RNA molecule can be readily controlled by altering the poly(A) polymerase reaction time. A 1760-base, 5′-capped RNA was incubated with 8 U of poly(A) polymerase under reaction conditions described in the A-Plus™ Poly(A) Polymerase Tailing Kit (CELLSCRIPT™ Madison, WI, USA). FIG. 1 shows the length of the 3′-poly(A) tail added to the 1760 base RNA under standard reaction conditions after 10, 30, and 60 minutes. The un-tailed 1760 base RNA is shown in the zero-minute gel lane. Incubation for 10 minutes yielded RNA with a poly(A)-tail length of approximately 200 bases; after 30 minutes the poly(A)-tail length was approximately 350-400 bases and after 60 minutes the reaction produced a poly(A)-tail length of >500 bases. Applicants performed experiments to independently evaluate the effects on polyadenylation using the components and the methods provided in the A-Plus™ Poly(A) Polymerase Tailing Kit (CELLSCRIPT™, Madison, WI, USA) when the following parameters were varied: (i) the amount poly A polymerase, (ii) the molar amount of RNA template, and (iii) the reaction volume; and (iv) the reaction time up to 60 minutes. Based on the results of those reactions (data not shown), it was found that:
A. The length of the poly(A)-tail was directly proportional to the amount of poly(A) polymerase in the reaction. The more poly(A) polymerase in the reaction, the longer the poly(A)-tail length.
B. The length of the poly(A)-tail was inversely proportional to the molar amount of RNA in the reaction. The less RNA present in the reaction, the longer the poly(A)-tail length.
C. The length of the poly(A)-tail was inversely proportional to the reaction volume. Reducing the reaction volume produced RNA with longer poly(A)-tails.
D. The length of the poly(A)-tail was increased with increased reaction time.

FIG. 2: Demonstration of berberine, in particular berberine hydrochloride, enhanced fluorescence of Poly A tails & PolyA oligos compared to SYBR-Gold™ Alone. The signal intensities of the PolyA 20-mer Marker Ladder, a 100-mer RNA ladder and a PolyA tail released from mRNA on denaturing PAGE gels stained with berberine, in particular berberine hydrochloride, +SYBR-Gold (left) or SYBR-Gold alone (right). Eight units of a poly(A) polymerase from an A-Plus™ Poly(A) Polymerase Kit (CELLSCRIPT, Madison, WI, USA) was incubated at 37° C. in a reaction mixture containing 60 micrograms of the 5′-capped RNA, 1× Reaction Buffer, and 1 mM ATP. One microliter aliquots of the reaction were removed at 0, 10, 30 and 60 minutes, and 0.1 μg of RNA from each time point was loaded and run on a 1% denaturing agarose gel with RNA markers, followed by gel staining with ethidium bromide, as shown in FIG. 2.

FIG. 3 and FIG. 4: Demonstration of example results of using different amounts of mRNA sample input (in pmol per 20 μL reaction). Additional exposure time on the scanner could rescue peaks for quantification down to 0.2 pmol. FIG. 3 shows a PAGE gel showing poly A tail length results using the indicated pmol input amount of RNA in 20-microliter RNase A reactions.

FIG. 4 shows a method of determining mRNA poly(A) tail length using the components of the kit as defined in the claims. It shows an ascending series of poly(A) tails including an encoded 120 nt tail (lane 2) along with a 136, 193, and 261 nt poly(A) tail respectively flanked by and compared with the 20-mer poly(A) ladder. 20-mer poly(A) ladder provides more accurate determination of tail lengths compared to mixed NTP ladders due to differential migration, as shown in FIG. 4.

FIG. 5: Representation of a comparison of the Poly(A) 20-mer marker ladder of the present invention with other RNA oligo ladders and different gel run times to verify ladder size. It is shown a 35-minute, 70-minute, and 120-minute gel run respectively. Each was run at 300V. Gel box was able to determine laddering peaks between 60-500 nt of ladder. IDT and Thermo ladders were used to verify ladder size.

FIG. 6: Said figure shows a 20-mer poly(A) ladder with 100 nt single stranded DNA marker for accurate size determination of poly(A) tails in gel electrophoresis-based assays. FIG. 6 demonstrates the use of PAGE-purified versus a desalted 100 nt oligo marker. The data show that desalted oligos contribute significant background and smearing of bands. PAGE purification is necessary to ensure good resolution and non-interference and streaking of peaks.

FIG. 7: Representation of an analysis done with denaturing polyacrylamide gel electrophoresis and a gel documentation system, as opposed to an LC-MS or CGE instrument.

FIG. 8 to FIG. 12: FIG. 8-12 show the use of LC MS analysis as a comparative benchmark to validate poly(A) tail length measurements obtained by an RNase A digestion and PAGE-based assay according to the present invention, wherein the comparative data demonstrates that the PAGE-based assay (also referred to in the description of FIG. 8-12 as “EZ-QC™ mRNA Poly(A) Tail Length Assay”) provides accurate sizing for tails within the LC MS measurable range and extends measurement capability beyond the LC MS upper limit of approximately 150 adenosines.

FIG. 8 demonstrates an analysis of the EZ-QC™ Poly(A) 20-mer Ladder with the EZ-QC™ mRNA Poly(A) Tail Length Assay. PAGE results and corresponding electropherogram showing lengths of the Poly(A) 20-mer Ladder included with the EZ-QC™ mRNA Poly(A) Tail Length Assay Kit. Lane 1: EZ-QC™ Poly(A) 20-mer Ladder. Lane 2: a variation of the Poly(A) 20-mer Ladder submitted for LC-MS. The 20-A peak is also present but was not captured here. The peaks are labelled 1-9 and their quantitation is denoted in the electropherogram.

FIG. 9 demonstrates LC-MS characterization of the Poly(A) 20-mer Ladder. LC-MS Analysis Summary of a Poly(A) 20-mer Ladder submitted for LC-MS. The 200-A peak and lengths greater than 200 were not detectable via LC-MS due to the limitations of the platform.

FIG. 10 demonstrates EZ-QC™ mRNA Poly(A) Tail Length Assay Analysis of a 20-mer RNA Substrate with Poly(A) Tail. PAGE results and electropherogram showing lengths of a sample processed with the EZ-QC™ mRNA Poly(A) Tail Length Assay Kit. Lane 1: EZ-QC™ Poly(A) 20-mer Ladder. Lane 2: a 20-mer RNA substrate with tail.

FIG. 11 demonstrates LC-MS Analysis of a 20-mer RNA Substrate with Poly(A) Tail. It shows LC-MS deconvoluted mass spectrum of 20-mer sample processed with the EZ-QC™ mRNA Poly(A) Tail Length Assay Kit and submitted for LC-MS testing.

FIG. 12 shows the compiled EZ-QC™ and LC-MS results. Comparable results were obtained between EZ-QC™ mRNA Poly(A) Tail Length Assay Kit and LC-MS for the same tailed RNA sample.

### DESCRIPTION OF THE INVENTION

One embodiment of the invention is a polyA size marker ladder comprising or consisting of a collection of single-stranded polyadenylic acid oligoribonucleotides (polyA oligos) of different lengths, each having a 5′-monophosphate and 3′-hydroxy group, wherein, the length in terms of the number of adenosines in the smallest of said polyA oligos in said collection is a first integer X selected from the group consisting of all integers from 10 to 30, and each next larger polyA oligo in said collection increases in number of adenosines by the same first integer X, and wherein the total number of adenosines in the largest poly(A) oligo in said collection is the first integer X multiplied by a second integer Y, wherein the second integer Y is the total number of different lengths of polyA oligos composing said polyA size marker ladder.

In one preferred embodiment of the polyA size marker ladder, the first integer X is selected from the group consisting of 20, 25 and 30.

In one preferred embodiment of the polyA size marker ladder, said first integer X is 20, and said ladder is a poly(A) 20-mer size marker ladder, wherein:
i) the smallest of said polyA oligos in said collection comprise numbers of adenosines selected from the group consisting of 20, 40, 60 and 80 adenosines, and
ii) the largest of said polyA oligos in said collection comprise numbers of adenosines are selected from the group consisting of 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, and ≥1,000 adenosines. In another preferred embodiment of the polyA size marker ladder, said first integer X is 25, and said ladder is a poly(A) 25-mer size marker ladder, wherein:
i) the smallest of said polyA oligos in said collection comprise numbers of adenosines selected from the group consisting of 25, 50, 75 and 100 adenosines, and
ii) the largest of said polyA oligos in said collection comprise numbers of adenosines are selected from the group consisting of 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and >1,000 adenosines.

In one preferred embodiment of the poly A size marker ladder, said marker ladder does not comprise any fragment comprising a nucleotide base selected from the group consisting of cytosine (C), guanine (G), thymine (T) and uracil (U).

In one preferred alternative embodiment of the poly A size marker ladder, said marker ladder comprises
i) at least one ssDNA reference tag oligo, said reference tag oligo comprising a nucleotide base selected from the group consisting of cytosine (C), guanine (G), thymine (T), and/or
ii) at least one ssRNA reference tag oligo, said reference tag oligo comprising a nucleotide base selected from the group consisting of cytosine (C), guanine (G), uracil (U).

In still other embodiments of said polyA size marker ladder, (A) said polyA size marker ladder further comprises at least one reference tag oligo comprising or consisting of a single-stranded DNA (ssDNA) oligo, and/or (B) said polyA size marker ladder further comprises at least reference tag oligo comprising or consisting of a single-stranded RNA (ssRNA) oligo;
wherein said reference tag oligo in A and/or B has at least one feature that enables it to be a reference tag oligo for identifying the size of at least one polyA oligo in said polyA size marker ladder based on where said at least one polyA oligo migrates during electrophoretic separation compared to said at least one reference tag oligo,
wherein said at least one feature of said at least one ssDNA reference tag oligo and/or of said at least one ssRNA reference tag oligo is selected from the group consisting of:
(i) It is labeled with a fluorescent, IR fluorescent, or visible dye covering the spectrum of 300 to 740 nm, or
(ii) It is labeled with biotin and is detectable with streptavidin bound to a fluorescent, IR fluorescent, or visible dye covering the spectrum of 300 to 740 nm, and/or
(iii) The amount of said at least one ssDNA reference tag oligo and/or of said at least one ssRNA reference tag oligo is 2-fold to 5-fold greater than the mass of the two closest polyA oligos with which it migrates during said electrophoretic separation.

In still other embodiments, the polyA size marker ladder further comprises:
(i) one or more reference tag oligos selected from the group consisting of at least one ssDNA reference tag oligo that has a number of nucleic acid bases that is an integer “Z-1” selected from the group consisting of: 25, 30, 45, 75, 90, 100, 150, 175, 200, 225, 275, 300, 350, 400, 450, 500, 540, 600, 630, 670, 710, 750, 790, 830, 880, 920, 970, and 1000; or
(ii) one or more ssRNA reference tag oligos that has a number of nucleic acid bases that is an integer “Z-2” selected from the group consisting of: 25, 30, 45, 75, 90, 100, 150, 175, 200, 225, 275, 300, 350, 400, 450, 500, 540, 600, 630, 670, 710, 750, 790, 830, 880, 920, 970, and 1000; or
(iii) comprises one ssDNA oligonucleotide of 100 nucleic acid bases in length that has a mass that is 2-fold to 5-fold greater than the 80-base and 100-base poly(A) oligos in said polyA size marker ladder.

Another embodiment of the invention is a kit or kit-of-parts comprising (a) RNase A, and (b) a size market ladder as described herein above.

In one embodiment of a kit or kit-of-parts of the invention, said at least one reference tag oligo serves as reference tag oligo for identifying the size of at least one polyA oligo in said a polyA size marker ladder based on the electrophoretic migration distance of said at least one reference tag oligo compared the electrophoretic migration distance of the closest polyA oligo in said size marker ladder, whether said reference tag oligo is in the same lane or a separate lane of a PAGE, agarose, or combination agarose+PAGE gel, or a capillary gel or matrix, cartridge matrix, or column matrix.

In one preferred embodiment, a kit or kit-of-parts of the invention further comprises:
a) an RNase A reaction buffer, preferably a 10× RNase A reaction buffer, and/or
b) RNase-free water.

In another preferred embodiment, a kit or kit-of-parts of the invention further comprising an electrophoresis gel, preferably an agarose or polyacrylamide gel, further preferably a 10% acrylamide, 8M urea, 10×TBE PAGE gel.

In particularly preferred embodiments of a kit or kit-of-parts of the invention, said kit or kit-of-parts further comprises berberine, in particular berberine hydrochloride, or a quaternary ammonium salt in the protoberberine group of benzylisoquinoline alkaloids that binds polyA and poly(A) oligos and enhances fluorescence of at least SYBR Gold and Oxazole-Gold cyanine dyes.

In another embodiments of a kit or kit-of-parts of the invention, said kit or kit-of-parts that further comprises a quaternary ammonium salt from the protoberberine group of benzylisoquinoline alkaloids further comprises coralyne. However, the present Applicants found that when electrophoretic separation was in a polyacrylamide gel, the background fluorescence was about higher and the binding of polyA and polyA oligos was lower using coralyne than using berberine, so berberine is preferred over coralyne.

In other embodiments of the invention, said kit or a kit-of-parts further comprises a poly(A) oligo-binding and/or staining dye selected from the group consisting of GelRed (Biotium), cyanine 5, ethidium, bromide, coralyne, berberine, in particular berberine hydrochloride, protoberberine, palmatine, jatrorrhizine, coptisine, berberrubine, harmine, harmaline, harmane, harmalol, tryptoline, amarastelline A, annuloline, serpentine, peperine and oxazole gold, wherein said poly(A) oligo-binding and/or staining dye is preferably berberine, in particular berberine hydrochloride.

In some embodiments of the invention, said kit or a kit-of-parts that comprises berberine, in particular berberine hydrochloride, further comprises or is used with a second dye that binds polyA and that is not berberine or a dye in the protoberberine group, wherein said second dye in combination with berberine results in higher fluorescence than when either dye is used alone.

In some preferred embodiments, said kit or kit-of-parts that comprises berberine, in particular berberine hydrochloride, further comprises or is used with SYBR-Gold or Oxazole Gold cyanine dye as a second dye.

In some particularly preferred embodiments, said second polyA-binding dye in or used with said kit or kit-of-parts is a concentrated solution, preferably 10,000× concentrated solution, of SYBR-Gold or Oxazole Gold cyanine dye in DMSO.

One preferred use of the polyA size marker ladder of the invention or the use of the kit or kit-of-parts is a method of assaying the length of a poly(A) tail of an mRNA comprising said poly(A) tail, wherein said mRNA is preferably an in-vitro synthesized or an in-vitro manufactured mRNA, and said method comprises the following steps:
a) digesting said mRNA comprising said poly(A) tail with RNase A,
b) parallel electrophoresis
   the RNase A digestion products of step a), and
   a polyA size marker ladder of any of the embodiments of the invention or any of the polyA size marker ladders of any of the kits or kits-of-parts in an electrophoretic separation instrument or device comprising a gel or a capillary tube, a column containing a gel or a sizing matrix, or a cartridge containing a gel or a sizing matrix, and
c) comparing
   the size of the poly(A) oligos of said polyA size marker ladder
   and optionally also comparing the size of the optional at least one reference tag oligo comprised by said polyA size marker ladder
   with the size of the poly(A) tails released from said mRNA in step a).

One preferred embodiment of the invention is the use of any of the polyA size marker ladders of any of the embodiments of the invention or any of the polyA size marker ladders of any of the kits or kits-of-parts of the invention, wherein, in step b), said RNase A digestion products and said polyA size marker ladder are electrophoresed on said electrophoresis gel in different lanes, wherein said gel is preferably a 10% acrylamide, 8M urea, 1×TBE PAGE gel.

Another preferred embodiment is the use of any of the polyA size marker ladders of any of the embodiments of the invention or any of the polyA size marker ladders of any of the kits or kits-of-parts of the invention, wherein, prior to step b), the RNase A digestion products of step a) and the polyA size marker ladder are heat-denatured, preferably at 65-70 degrees C. for 5 minutes in Stop/Loading Buffer.

Another preferred embodiment is the use of, wherein, following completion of said electrophoresis in step b), said gel is treated with a dye, preferably with SYBR-Gold and berberine, in particular berberine hydrochloride, and wherein said comparing in step c) is carried out by inspecting or analyzing the dye-treated gel using a fluorescent scanner, fragment analyzer, bioanalyzer, capillary electrophoresis instrument, or transilluminator.

Another embodiment of the invention is a method of using any of the poly A size marker ladders of any of the embodiments of the invention or any of the polyA size marker ladders of any of the kits or kits-of-parts of the invention to assay the length of a poly(A) tail of an mRNA comprising said poly(A) tail, wherein said mRNA is preferably an in-vitro synthesized or an in-vitro manufactured mRNA, said method comprising the followings steps:
a) digesting said mRNA comprising said poly(A) tail with RNase A,
b) parallel electrophoresis
   the RNase A digestion products of step a), and
   the poly A size marker ladder of any of any of the embodiments of the invention or the marker ladder of any of the embodiments of the kit or kit-of-parts on an electrophoresis gel or any other electrophoretic separation device or instrument, and
c) comparing
   the size of the poly(A) oligos of said polyA size marker ladder
   and optionally also comparing the size of the optional at least one reference tag oligo comprised by said polyA size marker ladder with
   the size of the poly(A) tails released from said mRNA in step a).

Another embodiment of the invention is the method described above, wherein, in step b), said RNase A digestion products and said polyA size marker ladder are electrophoresed on said electrophoresis gel in different lanes, wherein said gel is preferably a 10% acrylamide, 8M urea, 1×TBE PAGE gel.

Another embodiment of the invention is the method described above, wherein, prior step b), the RNase A digestion products of step a) and the polyA size marker ladder are heat-denatured, preferably at 65-70 degrees C. for 5 minutes in Stop/Loading Buffer.

One preferred embodiment of the invention is the method described above, wherein, following completion of said electrophoresis in step b), said gel is treated with a dye, preferably with both
(i) a dye selected from SYBR-Gold or Oxazole Gold cyanine dye, and
(ii) berberine, in particular berberine hydrochloride, or a quaternary ammonium salt from the protoberberine group of benzylisoquinoline alkaloids or a metabolite of any thereof that functions as a fluorescent enhancer of said dye selected in (i).

The compositions, kits, methods, and uses developed and described herein enable accurate, easy, fast, and reliable quantification of precise mRNA poly(A) tail lengths and size distributions. These methods directly measure the size and size distribution of the polyA tails that are released from the mRNA or other polyA-containing RNA following specific enzymatic digestion of the non-poly A portion of the mRNA or other RNA. The polyA product released following this enzymatic digestion is directly used for electrophoretic separation polyA size analysis, without use of any prior purification method of any kind, assuring that there have been no losses or modifications of the released polyA tail due to interactions with other materials or binding to other surfaces. This invention arose from the need for a simple, cost-effective solution that can be widely used in RNA research and development, mRNA manufacturing process development as well as for manufacturing in-process QC so that minor time or other process adjustments can be made during manufacturing to assure that the manufactured mRNA will comply with the poly A tail length specifications of the mRNA Quality Control (QC) Department.

The ease of use and high quality, accuracy and precision of the polyA tail length data obtained using those same compositions, kits, methods also enable the mRNA QC Department to use them to create and validate their use in its QC testing polyA tail length and size distribution specifications for each completed Master Batch Record of a finished mRNA product, such as an mRNA vaccine or therapeutic product. The precision, accuracy, consistency and clarity of such poly A tail length and size distribution QC data will save a lot of time for the people in the Quality Assurance (QA) Department in their review of the final Master Batch Record for such mRNA vaccine or therapeutic products.

### Definitions

A “PolyA Size Marker Ladder”, which is a composition of the present of the invention, herein means a size marker ladder comprising or consisting of a collection of single-stranded polyadenylic acid oligoribonucleotides (polyA oligos) of different lengths, each having a 5′-monophosphate and 3′-hydroxy group, that provides a standard by which the sizes of other polyadenylic acid molecules can be compared when separated based on size using an electrophoretic separation method; wherein, the length in terms of the number of adenosines in the smallest of said polyA oligos in said collection is a first integer X selected from the group consisting of all integers from 10 to 30, and each next larger polyA oligo in said collection increases in number of adenosines by the same first integer X, and wherein the total number of adenosines in the largest poly(A) oligo in said collection is the first integer X multiplied by a second integer Y, wherein the second integer Y is the total number of different lengths of polyA oligos composing said polyA size marker ladder.

A “Poly(A) Fluorescence Enhancer” herein means a biochemical substance that binds to polyadenylic acid (abbreviated as either “poly(A)” or “polyA”) oligoribonucleotides and polyribonucleotides, including the poly A tails that are present at the 3′-ends of most eukaryotic messenger RNA (mRNA) molecules, which biochemical substance, is capable of emitting fluorescent light when excited by certain defined wavelengths of light, and, in addition, is capable of enhancing the fluorence emitted by certain other fluorescent dye molecules that are also bound to those polyA molecules. This polyA fluorescence enhancement effect was observed during the work described in the present application, as shown by the data presented in FIG. 1. The data show that the fluorescent intensities when only SYBR-Gold cyanine dye was bound to poly A tails released from mRNA and to a collection of polyA oligos composing a polyA size marker ladder were much lower than the fluorescent intensities when both SYBR-Gold cyanine dye and the plant-derived alkaloid berberine, in particular berberine hydrochloride, were bound to polyA tails and the polyA oligos composing the polyA size marker ladders. Thus, berberine, in particular berberine hydrochloride, significantly enhanced the fluorescence emitted by the SYBR-Gold cyanine dye, thereby, greatly enhancing the sensitivity of the polyA tail length assay.

A “poly(A) polymerase” (“PAP”) means a template-independent RNA polymerase found in most eukaryotes, prokaryotes, and eukaryotic viruses that selectively uses ATP to incorporate AMP residues to 3′-hydroxylated ends of RNA. Since PAP enzymes that have been studied from plants, animals, bacteria and viruses all catalyze the same overall reaction (e.g., see Edmonds, M, Methods Enzymol., 181; 161-180, 1990), are highly conserved structurally (e.g., see Gershon, P, Nature Structural Biol. 7:819-821, 2000), and lack intrinsic specificity for particular sequences or sizes of RNA molecules if the PAP is separated from proteins that recognize AAUAAA polyadenylation signals (Wilusz, J and Shenk, T, Cell 52:221, 1988), purified wild-type and recombinant PAP enzymes from any of a variety of sources can be used for in vitro synthesis of polyadenylated RNAs (e.g., mRNAs) that are analyzed for polyA tail lengths and polyA size distributions using the compositions, kits, and the methods and uses of the present invention.

As used herein, a “T7-type” RNA polymerase” (RNAP) means T7 RNA polymerase (e.g., see Studier, F W et al., pp. 60-89 in Methods in Enzymology, Vol. 185, ed. by Goeddel, D V, Academic Press, 1990) or an RNAP derived from a “T7-type” bacteriophage, meaning a bacteriophage that has a similar genetic organization to that of bacteriophage T7. Examples of T7-type bacteriophages according to the invention include, but are not limited to Escherichia coli phages T3, phi I, phi II, W31, H, Y, A1, 122, cro, C21, C22, and C23; Pseudomonas putida phage gh-1; Salmonella typhimurium phage SP6; Serratia marcescens phages IV; Citrobacter phage ViIII; and Klebsiella phage No. 11 (Hausmann, Current Topics in Microbiology and Immunology 75:77-109, 1976; Korsten et al., J. Gen. Virol. 43:57-73, 1975; Dunn, et al., Nature New Biology 230:94-96, 1971; Towle, et al., J. Biol. Chem. 250:1723-1733, 1975; Butler and Chamberlin, J. Biol. Chem. 257:5772-5778, 1982), as well as mutant forms of such RNAPs (e.g., Sousa et al., U.S. Pat. No. 5,849,546; Padilla, R and Sousa, R, Nucleic Acids Res., 15: e138, 2002; Sousa, R and Mukherjee, S, Prog Nucleic Acid Res Mol Biol., 73:1-41, 2003).

Berberine-According to Wikipedia, “Berberine is a quaternary ammonium salt from the protoberberine group of benzylisoquinoline alkaloids, occurring naturally as a secondary metabolite in some plants including species of Berberis, from which its name is derived. Due to their yellow pigmentation, raw Berberis materials were once commonly used to dye wool, leather and wood. According to Chem Abstract Service, other names for berberine are: i) Benzo[g]-1,3-benzodioxolo[5,6-a]quinolizinium, 5,6-dihydro-9,10-dimethoxy-Umbellatine; ii) Berbinium, 7,8,13,13a-tetradehydro-9,10-dimethoxy-2,3-(methylenedioxy)-5,6-Dihydro-9,10-dimethoxybenzo[g]-1,3-benzodioxolo[5,6-a]quinolizinium; iii) Berberine; iv) Berbericine; v) Berberin; vi) Umbellatin; vii) Thalsine; viii) Majarine; and ix) Berberone; and biological sources of berberine are: Berberis vulgaris (barberry); Berberis aristata (tree turmeric); Berberis thunbergia, Fibraurea tinctoria; Mahonia aquifolium (Oregon grape); Hydrastis canadensis (goldenseal); Xanthorhiza simplicissima (yellowroot); Phellodendron amurense (Amur cork tree); Coptis chinensis (Chinese goldthread); Tinospora cordifolia; Argemone mexicana (prickly poppy); Eschscholzia californica (California poppy).

Chem Abstract Service Other Names for this Berberine Benzo[g]-1,3-benzodioxolo[5,6-a]quinolizinium, 5,6-dihydro-9,10-dimethoxy-Umbellatine Berbinium, 7,8,13,13a-tetradehydro-9,10-dimethoxy-2,3-(methylenedioxy)-5,6-Dihydro-9,10-dimethoxybenzo[g]-1,3-benzodioxolo[5,6-a]quinolizinium, Berberine, Berbericine, Berberin, Umbellatin, Thalsine, Majarine, Berberone

### Biological Sources

Berberis vulgaris (barberry), Berberis aristata (tree turmeric), Berberis thunbergii, Fibraurea tinctoria
Mahonia aquifolium (Oregon grape), Hydrastis canadensis (goldenseal), Xanthorhiza simplicissima (yellowroot), Phellodendron amurense (Amur cork tree), Coptis chinensis (Chinese goldthread), Tinospora cordifolia, Argemone mexicana (prickly poppy), Eschscholzia californica (California poppy), Berberine is usually found in the roots, rhizomes, stems, and bark.

SYBR-Gold dye (ThermoFisher/Invitrogen) or Oxazole Gold dye (Biotium) in combination with Berberine for staining polyA tails and said collection of polyA oligos composing said polyA size marker ladder following their electrophoresis on 10% acrylamide, 8M urea, 10×TBE PAGE gels under the conditions as taught in the present application.

RNAs to which the Compositions, Kits, Kits-of-Parts, and Methods and Uses of the Present Invention Apply

In general, the compositions, kits, kits-of-parts, and methods and uses of the present invention apply to and can be used to analyze the poly A tail length and size distribution of any 3′ poly A tail of a eukaryotic mRNA that is synthesized or manufactured in vitro or that is isolated from a natural source, and that comprises the following features or elements and can be represented by the following structure:

“5′-cap-5′ UTR-ATG-ORF-TAG, TAA, or TGA-3′ UTR-3′ polyA tail”,
wherein:
(1) One feature or element is a “5′ cap”, which means a structure at the 5′ end of the mRNA that is crucial for stability and translation initiation in eukaryotes. In humans and other mammals on most other animals, the 5′ cap structure comprises at least 2 nucleotides, including an N⁷-methylguanosine that is joined via its 5′-carbon to a triphosphate group that, in turn, is joined to the 5′-carbon of the first-transcribed nucleotide of the primary mRNA transcript. Such a capped transcript can be represented as m⁷G(5′)ppp(5′)N₁(pN)ₓ-OH(3′), or more simply, as m⁷GpppN₁(pN)ₓ, where m⁷G represents the 7-methylguanosine cap nucleoside, ppp represents the triphosphate bridge between the 5′ carbons of the cap nucleoside and the first nucleotide of the primary RNA transcript, and N¹(pN)ₓ-OH(3′) represents the primary RNA transcript, of which N₁ is the most 5′-nucleotide.
(2) One feature or element is a “5′ UTR”, which means a 5′ untranslated region of the mRNA comprising a sequence that is not translated into protein and that can contain regulatory elements that influence the translation of the mRNA.
(3) One feature or element is a “Start Codon”, which means a codon that marks the beginning of the protein coding sequence, which, in most cases, is ATG.
(4) One feature or element is an “ORF” or “Open Reading Frame”, which means the coding sequence of the mRNA that is translated into protein.
(5) One feature or element is a “Stop Codon”, which means a codon that marks the end of a protein coding sequence, which in most cases is at least one codon selected from the group consisting of TAG, TAA, and TGA. However, in some cases, a different Stop Codon is used.
(6) One feature or element is a “3′ UTR”, which means a 3′ untranslated region comprising a sequence at the 3′ end of the mRNA that is not translated into protein and that can have regulatory elements that influence the translation of the mRNA. If the mRNA has been isolated or purified from a natural source comprising or derived from a mammalian or other eukaryotic cells, the 3′ UTR probably has a polyadenylation signal sequence, at which site a multi-protein complex, including a cleavage/polyadenylation specificity factor, polyadenylate polymerase and other factors have cleaved and polyadenylated the products of RNA polymerase II. Often, but not always, the polyadenylation signal sequence is AAUAAA if the mRNA is derived from humans or other mammals, but this polyadenylation signal sequence is less common in plants and fungi.
(7) One feature or element is a “3′ polyA tail”, which means a string of adenosine nucleotides that is important for mRNA stability and translation of said mRNA, wherein:
(a) said mRNA that has been isolated or purified from a natural source comprising or derived from a mammalian or other eukaryotic cells, wherein said mRNA comprising said 3′ poly A tail has been added to the 3′ end of the mRNA during post-transcriptional processing in the nucleus of said cells and said mRNA that is analyzed comprises all of the features or elements of #(1) through #(6) above that apply to mRNA that has been isolated or purified from a natural source comprising or derived from a mammalian or other eukaryotic cells, including wherein said polyA tails from such natural source are interrupted or segmented by one or more nucleotides having a nucleic acid base other than adenine, as has been reported in the art (e.g., Y Liu et al, Poly(A) inclusive RNA isoform sequencing (PAIso-seq) reveals wide-spread non-adenosine residues within RNA poly(A) tails, Nature Commun 10, Article #5292, 2019); or
(b) said mRNA comprising said 3′ polyA tail has been synthesized or manufactured in vitro by IVT of a dsDNA template comprising a template strand that encodes all of the features or elements #(2) through #(6) plus a poly(dT) sequence that is joined thereto and that encodes said 3′ poly A tail feature or element #(7) of any desired length, wherein the is of the dsDNA IVT template that encodes the 3′ poly A tail, such that the 3′ poly A tail is joined to the 3′-end of the RNA product of IVT that encodes all of features #(2) through #(6); Note: if desired, mRNA comprising all of the features or elements of #(1) through #(7), including the 5′ cap feature of element #(1) can be synthesized by IVT by using a CleanCap® cap analog (TriLink) or an alternative cap analog from another commercial source during IVT; or
(c) said 3′ polyA tail has been synthesized or manufactured in vitro by IVT of a dsRNA template, wherein the template strand encodes all of features or elements #(2) through #(6), and then said RNA comprising all of said features or elements #(2) through #(6) has been 3′-polyadenylated using a poly(A) polymerase (e.g., using A-Plus™ Poly(A) Polymerase Tailing Kit, CELLSCRIPT™, Madison, WI, USA), and, if desired, mRNA comprising all of the features or elements of #(1) through #(7), including the 5′ cap feature or element #(1) can be synthesized by post-transcriptionally 5′-capping the RNA comprising all of the features or elements #(2) through #(6) to a cap 0 structure using a capping enzyme system and/or to a cap 1 structure using a capping enzyme and a cap1 2′-O-methyltransferase, e.g., using a ScriptCap™ m⁷G Capping System±ScriptCap™ 2′-O-Methyltransferase Kit, or ScriptCap™ Cap 1 Capping System (CELLSCRIPT™, Madison, WI, USA).

In addition, the compositions, kits, kits-of-parts, and methods and uses of the present invention also apply to analyzing the polyA tail lengths and size distributions of mRNAs in vitro-synthesized or isolated or purified from naturally occurring sources comprising cells, tissues or organisms, or derived from such sources. In some embodiments, the mRNAs comprising segmented or interrupted tails are synthesized in vitro.

In addition to being applicable for analyzing the poly A tail length and size distribution of poly A tails on any eukaryotic mRNA, the compositions, kits, kits-of-parts, and methods and uses of the present invention also apply to and can be used for analyzing the lengths and size distribution of:
(d) polyA stretches in any of the many types of naturally occurring and in vitro-synthesized RNA molecules that are currently known in the art or that are identified or made in the future, including, but not limited to:
   (i) circular ssRNA molecules encoding one or more proteins of interest for expression in a mammalian, human, animal, or other eukaryotic cell, including for a medical, therapeutic, prophylactic (e.g., vaccine), veterinary, or agricultural use; or
   (ii) naturally occurring in vitro-synthesized polyA tails or internal stretches of polyA in non-coding RNAs and long non-coding RNAs and in other RNAs encoded by the as-yet little-known and little-understood dark genomes of humans, plants and other eukaryotic and fungal organisms; or
   (iii) naturally occurring polyA tails that are interrupted by non-adenosine nucleotides (e,g, as described in Yusheng Liu, Poly(A) inclusive RNA isoform sequencing (PAIso-seq) reveals wide-spread non-adenosine residues within RNA poly(A) tails, Nature Communications 10, Article #5292, 2019); or
   (iv) in vitro-synthesized polyA tails that are segmented or interrupted by non-adenosine nucleotides or chemical spacers in order to decrease or slow the degradation of the poly A tails by cellular enzymes (e.g., nucleases), with the goal of extending the stability of the RNA molecule, and, if it is mRNA, to extend the life of the mRNA and its expression of the encoded protein (e.g., certain of the covid-19 spike protein vaccines used an interrupted or segmented tail for this purposes).
      Synthesis or Manufacture of mRNA Using a Process Comprising In Vitro Transcription

In some embodiments, the polyA tail that is analyzed for polyA tail length and size distribution using the compositions, kits, kits-of-parts, and methods and uses of the present invention is a 3′-polyA tail on an RNA that is synthesized or manufactured using a process that comprises in vitro transcription (IVT) of a dsDNA template that encodes the RNA or mRNA of interest using an RNA polymerase and ribonucleoside-5′-triphosphates (NTPs) in a buffered aqueous reaction mixture comprising magnesium cations. In some embodiments the NTPs comprise the four canonical NTPs consisting of ATP, UTP, GTP, and CTP. However, in some embodiments at least one modified NTP selected from the group consisting of pseudouridine-5′-triphosphate (ΨTP), 1-methylpseudouridine-5′-triphosphate (m¹Ψ′TP), 5-methoxyuridine-5′-triphosphate (mo⁵UTP), 5-methyluridine-5′-triphosphate (m⁵UTP), 2′-O-methyluridine-5′-triphosphate (UmTP or m^{2′-O}UTP), and 2-thiouridine-5′-triphosphate (s²UTP) is used for IVT in place of at least a portion of the uridine-5′-triphosphate (UTP). It has been shown that mRNA comprising Ψ, m¹Ψ, mo⁵U, m⁵U, m^{2′-O}U or s²U in place of at least a portion of the U nucleosides is significantly less immunogenic in term of its innate immunogenicity than the same mRNA comprising uridine as can be detected, for example, by measuring less secretion of IFN-α or TNF-α cytokine from human dendritic cells (DCs) at a defined time after being transfected with mRNA comprising a modified uridine nucleosides compared to the amount of the same cytokine secreted from the same number of human DCs at the same defined time after being transfected with the same amount of mRNA comprising uridine (see WIPO PCT Patent Application No. WO2007024708A2; K Karikó, et al, Nucleic Acids Res, 39 (21), November 2011, e142, https://doi.org/10.1093/nar/gkr695). The speed of development and the prophylactic effectiveness of the vaccines developed by Moderna and by BioNTech and Pfizer that contained m¹Ψ-modified mRNA encoding the covid-19 spike protein clearly demonstrated the benefit of the modified mRNA technology, for which Doctors Katalin Karikó and Drew Weissman were awarded the 2023 Nobel prize in Physiology or Medicine. It also inspired many people in academic, biotech and biopharm organizations to enter, explore and innovate new ideas and continual technological improvements and rapid growth in many areas of mRNA prophylactics and therapeutics. The benefits of this modified mRNA technology are also supported by public disclosures from public companies. For example, CureVac confirmed in public press releases that that their initial covid mRNA vaccine during the pandemic was less effective than the m¹Ψ-modified mRNA vaccines and that they were working on new mRNA modalities that seem to involve modified mRNAs.

In some embodiments, the polyA tail that is analyzed for polyA tail length and size distribution using the compositions, kits or kit-of-parts, methods and uses of the present invention is a 3′-polyA tail on an RNA that is synthesized or manufactured using a process that comprises IVT, wherein, the RNA is 5′-capped co-transcriptionally by including a cap analog in the IVT reaction mixture.

In some embodiments, the polyA tail that is analyzed for poly A tail length and size distribution using the compositions, kits or kit-of-parts, methods and uses of the present invention is a 3′-polyA tail on an RNA that is synthesized or manufactured using a process that comprises IVT, wherein, the RNA is 5′-capped using a cap analog that has been made using capping enzyme system and a modified nucleotide as a substrate, as taught in WIPO PCT Patent Application No. WO2007120863A2 and U.S., European and Canadian patents based thereon. Eukaryotic mRNA is a complex molecule comprising, in a 5′ to 3′ direction, a 5′-untranslated region (5′ UTR), a start codon (usually ATG) is the start codon, a coding region, a stop codon (usually TAG, TAA, or TGA), and a 3′-untranslated region (3′-UTR) or that usually a ssRNA molecule encoded by a gene. RNA polymerase transcribes the template strand of the dsDNA gene to form a pre-mRNA that is cleaved. The transcribed RNA is processed in that is synthesized by a transcription enzyme that is encoded by a gene and that is translated into protein that corresponds to the genetic sequence of a gene and is read by a ribosome in the process of synthesizing a protein. Naturally occurring mRNA in cells of eukaryotes almost universally have a 5′ cap comprising N⁷-methylguanosine that is joined via its 5′-carbon to a triphosphate group that is, in turn, joined to the 5′-carbon of the most 5′-nucleotide of the primary RNA transcript that is and a 3′ poly A tail comprising a stretch of adenosines that is synthesized by an RNA polymerase have in some embodiments, the polyA tail that is analyzed for polyA tail length and size distribution using the compositions, kits, methods and uses of the present invention, that has been synthesized or manufactured in vitro as discussed above is further poly adenylated.

In some embodiments, the polyA tail that is analyzed for poly A tail length and size distribution using the compositions, kits, methods and uses of the present invention, a dsDNA template is designed so its template strand has a template-encoded polyA tail encoded by poly(T) stretch genetically engineered into the DNA template strand. One advantage of encoding the 3′ polyA tail in the dsDNA template is that it is not necessary to perform another enzymatic reaction using a Poly A Polymerase and ATP after the IVT reaction. Another advantage of co-transcriptional poly A tailing is that the dsDNA template can be designed to encode a pre-determined tail length. If the dsDNA template is a plasmid that is maintained in a bacterial host like E. coli, one disadvantage of co-transcriptional polyA tailing is that polyA tails that dsDNA plasmid template that encode poly A tails that are longer than about 50-60 adenosines are genetically unstable and the poly template decreases in size. Applicants have observed that mRNA from almost every colony of E. coli cells that grew when a cell bank for a dsDNA plasmid encoding a polyA tail with >100 A's was plated out had a different polyA tail length. Some labs have tried to solve the problem of tail-length instability of plasmids with long tails by inserting other deoxy-nucleotides at intervals to interrupt the encoded adenosines in the tail. Applicants do not have experience with interrupted tails. Another alternative to solve the problem of plasmid instability is to not maintain and replicate the dsDNA templates for RNAs encoding open reading frames for proteins in plasmids in bacterial hosts. There is also interest in the field to generate DNA templates for IVT that do not require fermentation of bacteria and isolation and maintenance of plasmid. One possible method is rolling circle amplification, which has been used for diagnostics and many other applications for decades.

In some other embodiments, the poly A tail that is analyzed for polyA tail length and size distribution using the compositions, kits, methods and uses of the present invention is added to the IVT-RNA post-transcriptionally using a poly A polymerase.

### Methods for 3′-Polyadenylation Using a Poly(A) Polymerase

The invention also provides kits and methods for obtaining modified-nucleotide-capped RNA that has a poly(A) tail. This can be beneficial because capped RNA that has a poly(A) tail is more stable and is translated with higher efficiency in vivo, and sometimes in vitro, than the same RNA that lacks a poly(A) tail. Thus, in some embodiments, the kit further comprises poly(A) polymerase. In some embodiments of the invention, the method further comprises the step of contacting the modified-nucleotide-capped RNA, having either a cap 0 or a cap 1 structure, with poly(A) polymerase and ATP under conditions wherein modified-nucleotide-capped RNA having a poly(A) tail is synthesized. In some embodiments, the length of the poly(A) tail synthesized is about 30 nucleotides. In other embodiments, the length of the poly(A) tail synthesized is about 30 nucleotides to about 100 nucleotides. In other embodiments, the length of the poly(A) tail synthesized is about 100 nucleotides to about 200 nucleotides. In still other embodiments, the length of the poly(A) tail synthesized is about 200 nucleotides to about 400 nucleotides. In some embodiments, the length of the poly(A) tail synthesized is greater than 400 nucleotides. In some embodiments wherein the modified-nucleotide-capped RNA is translated in vitro in cell-free extracts, the modified-nucleotide-capped RNA lacks a poly(A) tail. Without limitation, the poly(A) polymerase can be selected from the group consisting of Escherichia coli poly(A) polymerase and yeast poly(A) polymerase. In some embodiments the poly(A) polymerase is recombinant poly(A) polymerase encoded by the E. coli pcnB gene. Methods for polyadenylating RNA using a poly(A) polymerase are well known in the art and kits for such purpose are commercially available. For example, RNA can be polyadenylated using A-Plus™ poly(A) polymerase tailing kit (CELLSCRIPT, Madison, WI, USA) according to the directions provided with the kit.

Poly(A) Polymerase enables rapid, efficient, and controlled addition of a poly(A)-tail to the 3′-end of any RNA in vitro. The presence of a poly(A)-tail at the 3′-end of an RNA molecule can have an important and positive impacts on RNA, including increased stability and enhanced translation after transfection or microinjection into eukaryotic cells (Drummond, D. R. et al., J. Cell. Biol. 100 (4), 1148, 1985; Galili, G. et al., J. Biol. Chem. 263 (12), 5764, 1988; and Belasco, J and Brawerman, G. Control of Messenger RNA Stability, Academic Press, San Diego, CA, 1993.

Poly(A) Polymerase uses ATP as a substrate for template-independent addition of adenosine monophosphate to the 3′-hydroxyl termini of RNA molecules (Gething, M. et al., Nature 287 (5780), 301, 1980). Here, we demonstrate that the length of the poly(A)-tail added to the 3′-end of an in vitro transcribed RNA can be easily controlled using a post-transcriptional in vitro polyadenylation reaction with poly(A) polymerase and ATP in a buffered reaction mixture.

### Methods and Results

### In Vitro Transcription and Purification of a 5′-Capped RNA Template

A 1760-base, 5′-capped RNA analog (m⁷G [5′]ppp[5′]G) transcript was produced from a linearized DNA template using a standard 20-microliter in vitro transcription reaction with T7 RNA polymerase from a AmpliCap-MAXIM T7 High Yield Message Maker Kit (CELLSCRIPT, Madison, WI, USA). The completed AmpliCap-MAX reaction was treated with 1 unit of RNase-Free DNase I to remove the DNA template, and the 5′-capped RNA was purified by addition of 20 microliter of 5M ammonium acetate, followed by incubation on ice for 15 minutes and centrifugation at 10,000×g for 15 minutes. The ammonium acetate selectively precipitates RNA while leaving most of the DNA, protein and unincorporated NTPs in the supernatant. The resulting pellet containing the capped-RNA transcript was washed with cold 70% ethanol, dried, and resuspended in 40 microliter of sterile RNase-free water. The yield of the RNA was measured at A260 and the quality of the 5′-capped RNA was confirmed by 1% denaturing agarose gel electrophoresis.

Poly(A)-Tailing of RNA with Poly(A) Polymerase

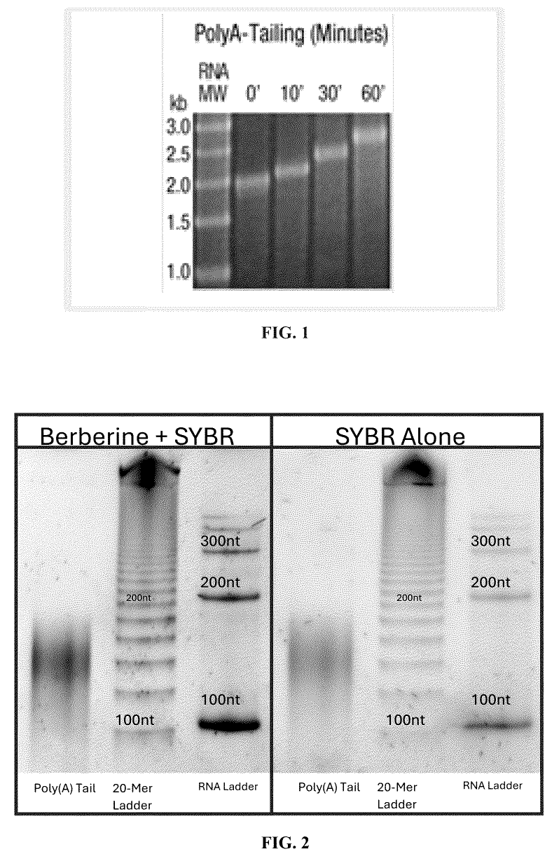

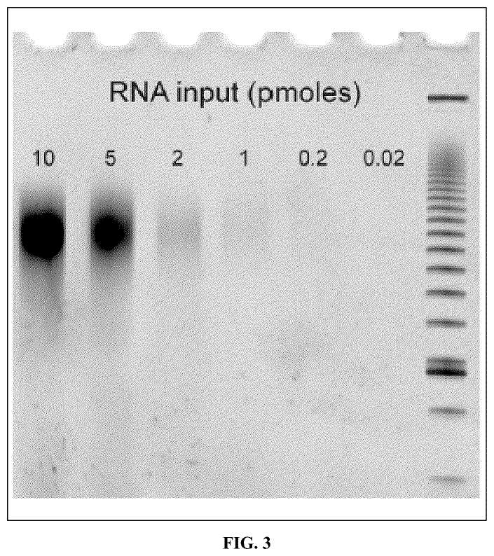

Eight units of a poly(A) polymerase from an A-Plus™ Poly(A) Polymerase Kit (CELLSCRIPT, Madison, WI, USA) was incubated at 37° C. in a reaction mixture containing 60 micrograms of the 5′-capped RNA, 1× Reaction Buffer, and 1 mM ATP. One microliter aliquots of the reaction were removed at 0, 10, 30 and 60 minutes, and 0.1 μg of RNA from each time point was loaded and run on a 1% denaturing agarose gel with RNA markers, followed by gel staining with ethidium bromide, as shown in FIG. 2.

An mRNA Poly(A) Tail Length Assay Kit

One embodiment of the invention is an mRNA Poly(A) Tail Length Assay Kit, wherein said Kit comprises or consists of:

RNase A in 50% glycerol, 50 mM Tris-HCl, pH 7.5, 100 mM NaCl,
1 mM dithiothreitol (DTT), 0.1 mM EDTA and 0.1% Triton ® X-100.
10X RNase A Reaction Buffer
0.6M Tris-HCl, pH 8.0, 1M NaCl, 0.1M EDTA.
Stop/Loading Buffer
95% formamide, 10 mM EDTA, pH 7.5, 0.01% Bromophenol Blue
& 0.01% Xylene Cyanol.
Berberine Poly(A) Fluorescence Enhancer, 100X
Protect the tube from exposure to light!
PolyA 20-Mer Size Marker Ladder
Ready-to-Load: Supplied in Stop/Loading Buffer for accurate size
determination up to 500 nucleotides
RNase-Free Water

Key Highlights of the mRNA Poly(A) Tail Length Assay Kit of the Invention:

The first step of the process for assaying the poly A tail length of an mRNA, such as an in vitro-synthesized or manufactured RNA or mRNA, is to use the provided RNase A enzyme, 10× RNase A Reaction Buffer and RNase-Free Water and to follow the Procedure for the RNase A Treatment Step to digest non-polyA regions of mRNA and release the polyA tails from each of the 5-picomole samples mRNA molecules being analyzed for poly A tail length. To do this, the analyst must first calculate what the 5-picomole sample amount is in terms of micrograms for each RNA or mRNA sample you wish to analyze. An example of how this is calculated for an RNA having 1,700 nucleotides is given below: pmole/microgram ssRNA=10⁶/the estimated length of the mRNA in nucleotides×330 E.g., for a 1,700 nt RNA: 10⁶/(1,700×330)=1.8 μmol/microgram

The user may substitute the number of nucleotides for each RNA being analyzed to calculate the number of pmol/microgram: then calculate the number of micrograms of that RNA needed to obtain the 5 picomoles needed for each RNase A Treatment.

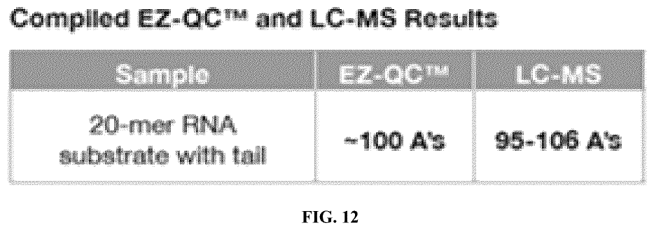

Once the analyst has set up all the RNase A 10-microliter reactions and control reactions, the next step is to incubate those reactions for 2 hours at 37° C. The RNase A treatment is stopped by adding 10 microliters of the provided Stop/Loading Buffer, which contains xylene cyanol and bromophenol blue dyes for tracking progress of electrophoresis of the samples on the 10% acrylamide, 8M urea, 10×TBE denaturing PAGE gel.
There is no need to enrich for or purify the poly(A) tail after RNase A treatment prior to loading the samples in the respective wells of the denaturing PAGE gel. When the PAGE gel is ready and has been prepared by blowing out the wells immediately after comb removal, pre-running the gel at 300 volts constant voltage for 20-30 minutes, and blowing out the excess urea from the wells just prior to sample loading, each RNase A-treated RNA sample and the PolyA Size Marker Ladder samples should be heated at 65-70 degrees C. for 5 minutes just prior to gel loading.
The RNase A-treated mRNAs should be five picomoles of each RNase A-digested mRNA can be loaded directly into a well of the denaturing PAGE gel, and then, following, electrophoresis and staining of the gel with SYBR-Gold™, an unsymmetrical cyanine dye, and the provided berberine fluorescence enhancer, poly(A) tail lengths can be read visually or using a gel scanner, saving much time and resources.
Use of a PolyA Size Marker Ladder comprising a collection of all 20-mer poly(A) oligos covering the full poly(A) tail size range from 20-40 adenosines up to about 600 adenosines, permit precise sizing of polyA tail lengths, (which should give a more accurate result than sizing the poly(A) tail by comparison to a ladder of mixed base composition.
Applicants found that staining gel with both berberine, in particular berberine hydrochloride, and SYBR Gold and produced more intense staining than either of these compounds did alone (FIG. 1), enabling polyA tail lengths to be determined with only 5 picomoles of each RNase A-treated mRNA sample input. (FIG. 12). In addition to berberine, Applicants have also evaluated the proto-berberine coralyne as a potential evaluated multiple. Some signal could be used but the coralyne gave a higher background signal when used in combination with SYBR Gold Dye (ThermoFisher) and therefore produced data that was more difficult to interpret than berberine in combination with SYBR Gold Dye. Therefore, berberine, in particular berberine hydrochloride, is preferred.

Analysis is done by visually inspecting or using a scanner, Syngene G-Box or other gel documentation system to quantify the mRNA polyA tail samples and the PolyA Size Marker Ladder following denaturing PAGE electrophoresis as opposed to an LC-MS or CGE instrument.

### Method of Use

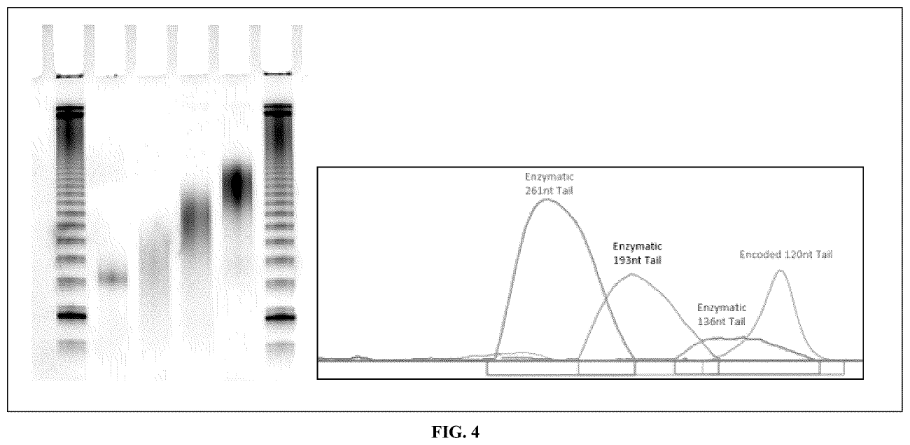

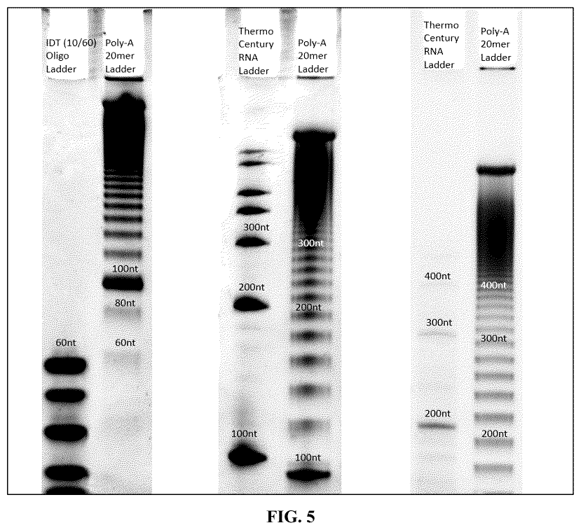

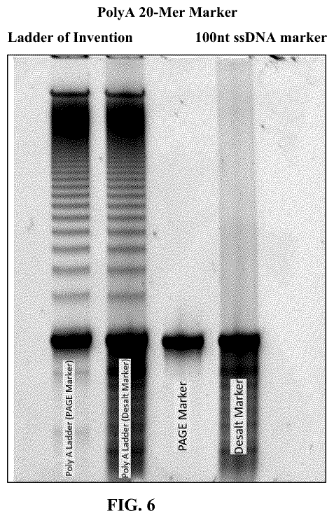

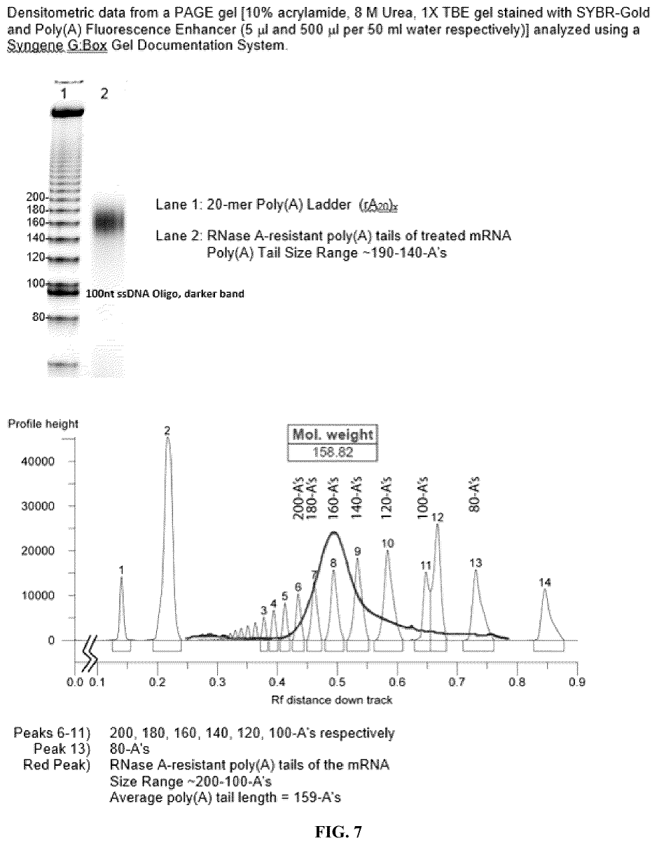

The kit utilizes a simplified approach compared to existing methods:
1. Sample preparation: The poly(A) mRNA isolation from the source material.
2. RNase A digestion: Selective degradation of non-poly(A) RNA regions.
3. Gel electrophoresis: Separation of poly(A) tails using PAGE. Samples can be directly loaded on gel without having to enrich post-digestion products.
4. Visualization: Enhanced fluorescence detection using berberine, in particular berberine hydrochloride.
5. Size determination: Comparison with the provided 20-mer poly(A) ladder. 20-mer poly(A) ladder provides more accurate determination of tail lengths compared to mixed NTP ladders due to differential migration FIG. 4.

### Applications of the Kit

The mRNA Poly(A) Tail Length Assay Kit can be used for:
1. Quality control of mRNA samples in research laboratories.
2. Assessment of mRNA stability and degradation in various experimental conditions.
3. Evaluation of poly(A) tail length changes in response to cellular stimuli or genetic modifications.
4. Quality control in mRNA-based therapeutic development and production.
5. Analysis of wide range of long poly(A) tails up to 500 adenosines or longer.

### Advantages and Improvements

1. Simplicity: The kit eliminates the need for complex PCR or sequencing steps.
2. Accessibility: Requires only basic gel electrophoresis equipment and gel scanners that are accessible in most governmental and academic research laboratories.
3. Cost-effective: Reduces the need for expensive sequencing or specialized instruments.
4. Accuracy: 20-mer poly(A) ladder provides precise size determination.
5. Enhanced visualization: berberine, in particular berberine hydrochloride, improves the detection of poly(A) tails, which can be challenging to visualize with standard methods. See FIG. 1.
6. Rapid results: The assay can be completed in a shorter time compared to sequencing-based methods.
7. Extended size range: The inclusion of an extended poly(A) ladder allows for quantitative measurement of tails between 60-500 nucleotides long, enabling the analysis of a wider range of poly(A) tail lengths.

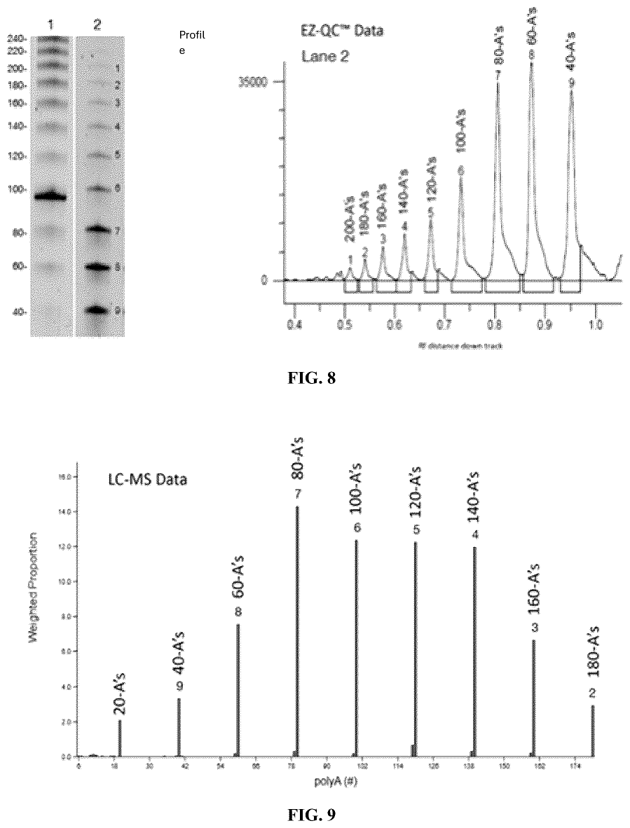

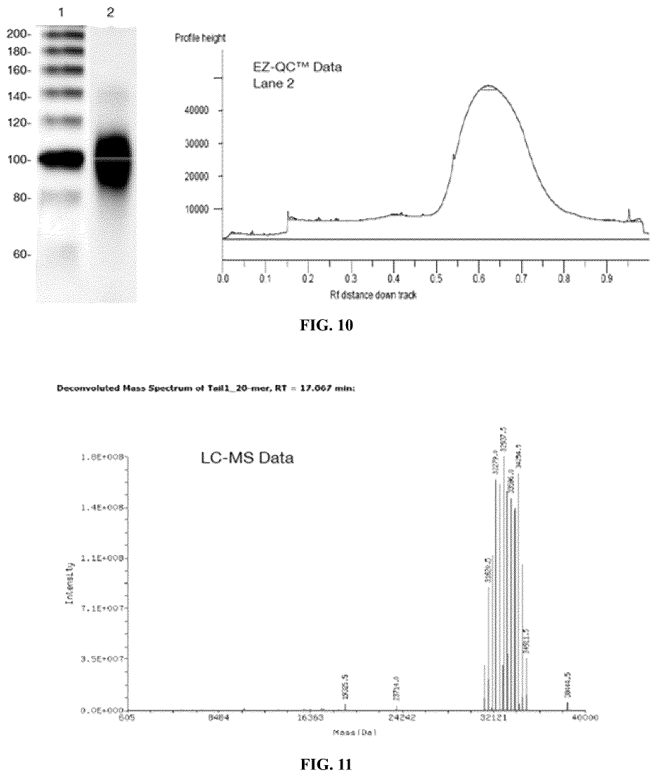

### ASSAY OF RNA POLY (A) TAIL LENGTH as Shown in FIG. 8-12

### LC-MS Benchmarking for Assay of RNA Poly(A) Tail Length

This Example provide use of LC-MS analysis as a comparative benchmark to validate poly(A) tail length measurements obtained by an RNase A digestion and PAGE-based assay, wherein the comparative data demonstrates that the PAGE-based assay provides accurate sizing for tails within the LC-MS measurable range and extends measurement capability beyond the LC-MS upper limit of approximately 150 adenosines. While various LC-MS methods are constrained to analyzing maximum Poly(A) tail lengths of just 120-150 bases, the EZ-QC™ mRNA Poly(A) Tail Length Assay exceeds those capabilities. The EZ-QC™ mRNA Poly(A) Tail Length Assay enables assessment of full-length mRNA with tails up to 300 adenosines, and with minor adjustments to gel running conditions, tails with up to 400 adenosines.

FIG. 8-12 relate to a method for determining and authenticating poly(A) tail length of mRNA, comprising:
1. digesting the mRNA with an RNase A reagent under conditions that preserve the 3′ poly(A) tail
2. resolving the poly(A) tail by polyacrylamide gel electrophoresis (PAGE) alongside a calibrated poly(A) ladder.
3. staining the gel with a fluorescent nucleic acid dye and a poly(A) fluorescence enhancer to visualize the tail.
4. determining the poly(A) tail length by comparison to the ladder.
5. performing liquid chromatography-mass spectrometry (LC-MS) analysis on the same sample to obtain reference measurements within the LC-MS measurable range; and
6. correlating the gel-based measurements with LC-MS data to authenticate results and document extended measurement capability beyond the LC-MS upper limit; wherein the method enables accurate sizing of poly(A) tails up to at least 300 adenosines under standard conditions and up to at least 400 adenosines under adjusted conditions.

The following key points are of relevance:
LC-MS as an authenticity benchmark:
   LC-MS is positioned as the recognized industry standard for poly(A) tail measurement, serving as a reference to validate the PAGE-based assay results.
Superiority of the inventive method:
   The PAGE-based assay not only matches LC-MS accuracy within its measurable range but extends capability beyond LC-MS's upper limit of ~150 adenosines, reaching up to 300-400 adenosines.
Integrated validation workflow:
   The inventive method explicitly incorporates LC-MS comparison as part of the process to authenticate results and document extended measurement capability.
Workflow advantages:
   Enables accurate sizing without LC-MS limitations, while maintaining simplicity and scalability for routine use.

### Procedures

A sample was submitted to Novatia, LLC, for the purpose of characterization of an mRNA using their LC-MS platform. Novatia utilizes liquid chromatography coupled to high resolution mass spectrometry and novel deisotoping and charge deconvolution software for characterization of mRNA caps and Poly(A) tails. 3′-end polyadenylation is a critical modification that influences mRNA stability and expression levels in eukaryotic cells with longer tails generally providing increased stability and translation efficiency. 3′-poly(A) tails can be added co-transcriptionally when encoded by template or post-transcriptionally via enzymatic addition with poly(A) polymerase. The EZ-QC™ mRNA Poly(A) Tail Length Assay employs EZ-QC™ RNase A to digest the mRNA molecule but leave the poly(A) tail intact. In conjunction with the EZ-QC™ Poly(A) 20-mer Ladder, the poly(A) tail length can be determined using PAGE followed by staining with SYBR™ Gold plus Poly(A) Fluorescence Enhancer. A minimum of 5 picomoles was required for EZ-QC™ mRNA Poly(A) Tail Length Assay, whereas 150-250 picomoles were required for LC-MS analysis.

Both the EZ-QC™ mRNA Poly(A) Tail Length Assay and the LC-MS method required identical upfront sample processing steps. After this shared initial workflow, their downstream requirements diverged. The EZ-QC™ mRNA Poly(A) Tail Length Assay generated material that was immediately suitable for electrophoretic analysis without the need for additional cleanup. In contrast, samples designated for LC-MS analysis required further purification using the Monarch Spin RNA Cleanup Kit to remove residual probe, buffer salts, and protein contaminants such as RNase H enzyme, all of which can interfere with mass spectrometric performance. Although purification was not necessary for samples analyzed solely by the EZ-QC™ assay, these samples were also purified to enable consistent, side-by-side comparison with LC-MS. Additionally, no electrophoresis-associated dyes were added to any samples prepared for LC-MS analysis.

The LC-MS method used in this study was HRMS_LCMS_PolyAtail, which required a specialized chromatography column (Clarity Oligo-xt, 2.1×50 mm). In contrast, the EZ-QC™ mRNA Poly(A) Tail Length Assay did not require any specialized column hardware. The cost of LC-MS analysis per sample was approximately 4-10 times higher than testing with the EZ-QC™ assay. Additionally, the EZ-QC™ assay allowed throughput of up to 10-15 samples per run, whereas LC-MS accommodated only one sample per run. LC-MS also required a dedicated system-suitability control for each run, a requirement not needed for the EZ-QC™ assay.

Both an EZ-QC™ Poly(A) 20-mer Ladder and an approximately 100-adenosine Poly(A) tail were evaluated side-by-side using both methods. The two platforms produced comparable results: Poly(A) ladders yielded similar size distributions, and the 100-mer Poly(A) tail showed highly consistent measurements across methods, with less than 6% difference in the reported tail lengths.

The EZ-QC™ mRNA Poly(A) Tail Length Assay did not require prior knowledge of the expected molecular weight or length of the target mRNA. Instead, Poly(A) tail fragments were resolved and quantified using standard electrophoretic separation and staining procedures described in the assay protocol. In contrast, LC-MS analysis required advance knowledge of the molecular weights and relative abundances of the mRNA-derived species to enable accurate identification and interpretation. This requirement was not necessary for the EZ-QC™ assay, which could be applied to both known and unknown samples.

From an instrumentation standpoint, the EZ-QC™ assay relied only on benchtop electrophoresis equipment and a gel imager, typically costing approximately $5,000-$10,000. LC-MS analyses performed on an Orbitrap mass spectrometer-required instrumentation costing several hundred thousand dollars to more than $1 million. The Orbitrap platform also required routine preventive maintenance, whereas electrophoretic equipment and gel imagers required minimal upkeep.

The operational complexity of the platforms differed substantially. The EZ-QC™ assay required only basic laboratory skills with no specialized training for data acquisition or interpretation. LC-MS workflows, however, required specialized training in instrument setup, operation, and data analysis software, particularly given the complexity of spectral outputs and the overlap in mass profiles of different molecules.

Workflow logistics also favored the EZ-QC™ assay. Testing could be performed entirely in-house, whereas LC-MS analysis often required outsourcing, vendor account setup, procurement coordination, and sample shipment. LC-MS also involved extensive upfront instrument preparation and specialized software knowledge. Turnaround time for the EZ-QC™ assay was typically one day, compared with 1-5 days for LC-MS, excluding shipping time.

All these distinctions, including workflow requirements, instrumentation needs, cost considerations, expertise levels, and turnaround times are summarized in the accompanying table for ease of comparison.

| Factor | EZ-QC ™ Assay Kits | LC-MS |
|---|---|---|
| Length of mRNA | No max length | <200 A's is the max fragment length to achieve |
| for Poly(A) | restrictions | optimal analysis |
| tail analysis | | |
| Quantity of | 5 picomoles and up for | 30-50 μl of a 5 μM solution = 150-250 |
| mRNA | Poly(A) tail length kit | picomoles total required for each QC analysis. |
| Turnaround time | Same day | >1-5 days, not including shipping time |
| Cost per sample | 4-10 samples for the cost | 1X LC-MS sample cost |
| | of 1X LC-MS sample | |
| Number of | 10-15 samples per gel, | 1 sample per run |
| samples per run | scalable based on needs | |
| Controls required | A 20-mer Poly(A) ladder | System suitability controls. Essential to provide |
| | is included | additional controls to run alongside unknowns. |
| Expected | Not required | Yes, so the software can assign expected |
| molecular weight | | molecular weights and relative abundance. |
| or length of target | | |
| needed | | |
| for analysis | | |
| Equipment | Benchtop electrophoresis | LC-MS platform |
| required | and gel imager | |
| Training required | Basic lab techniques | Yes, specialized training is required for using |
| | | the software, interpreting data and using the |
| | | equipment. Many molecules have similar |
| | | profiles, making analysis difficult. |
| Flexibility of | Process and analyze | Requires possible outsourcing of testing, |
| testing | samples when convenient | account setup, and scheduling of shipment on |
| | | dry ice. Requires up-front prep of platform and |
| | | knowledge of the analysis software and data |
| | | output. |

### REFERENCES

1. RC Yadav, et al., Berberine a strong polyriboadenylic acid binding plant alkaloid: spectroscopic, viscometric, and thermodynamic study, Bioorganic & Medicinal Chem., 13, 165-174, 2005.
2. Md. Maidul Islam, et al., Binding of 9-O-(w-amino) alkyl analogues of the plant alkaloid berberine to poly(A): insights into self-structure induction.
3 O. P. Ceninkol & N. V. Hud, Molecular recognition of poly(A) by small ligands: an alternative method of analysis reveals nonmolar, cooperative and shape-selective binding, Nucleic Acids Res. 37, 611-621, 2009.

1. A polyA size marker ladder comprising a collection of single-stranded polyadenylic acid oligoribonucleotides (polyA oligos) of different lengths, each having a 5′-monophosphate and 3′-hydroxy group, wherein, the length in terms of the number of adenosines in the smallest of said polyA oligos in said collection is a first integer X selected from the group consisting of all integers from 10 to 30, and each next larger polyA oligo in said collection increases in number of adenosines by the same first integer X, and
wherein the total number of adenosines in the largest poly(A) oligo in said collection is the first integer X multiplied by a second integer Y, wherein the second integer Y is the total number of different lengths of polyA oligos composing said polyA size marker ladder.

2. The polyA size marker ladder of claim 1, wherein the first integer X is selected from the group consisting of 20, 25 and 30.

3. The polyA size marker ladder of claim 1, wherein said first integer X is 20, and said ladder is a poly(A) 20-mer size marker ladder, wherein
i) the smallest of said polyA oligos in said collection comprise numbers of adenosines selected from the group consisting of 20, 40, 60 and 80 adenosines, and
ii) the largest of said polyA oligos in said collection comprise numbers of adenosines are selected from the group consisting of 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, and ≥1,000 adenosines.

4. The polyA size marker ladder of claim 1, wherein said first integer X is 25, and said ladder is a poly(A) 25-mer size marker ladder, wherein:
i) the smallest of said polyA oligos in said collection comprise numbers of adenosines selected from the group consisting of 25, 50, 75 and 100 adenosines, and
ii) the largest of said polyA oligos in said collection comprise numbers of adenosines are selected from the group consisting of 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and >1,000 adenosines.

5. The poly A size marker ladder of claim 1, wherein said marker ladder does not comprise any fragment comprising a nucleotide base selected from the group consisting of cytosine (C), guanine (G), and uracil (U).

6. The poly A size marker ladder of claim 1, wherein said marker ladder comprises
i) at least one ssDNA reference tag oligo, said reference tag oligo comprising a nucleotide base selected from the group consisting of cytosine (C), guanine (G), thymine (T), and/or
ii) at least one ssRNA reference tag oligo, said reference tag oligo comprising a nucleotide base selected from the group consisting of cytosine (C), guanine (G), uracil (U).

7. The polyA size marker ladder of, wherein:
A. said polyA size marker ladder further comprises at least one reference tag oligo comprising or consisting of a single-stranded DNA (ssDNA) oligo, and/or
B. said polyA size marker ladder further comprises at least reference tag oligo comprising a single-stranded RNA (ssRNA) oligo;
wherein said reference tag oligo in A and/or B has at least one feature that enables it to be a reference tag oligo for identifying the size of at least one polyA oligo in said polyA size marker ladder based on where said at least one polyA oligo migrates during electrophoretic separation compared to said at least one reference tag oligo.

8. The polyA size marker ladder of claim 7, wherein at least one feature of said at least one ssDNA reference tag oligo and/or of said at least one ssRNA reference tag oligo is selected from the group consisting of:
(i) It is labeled with a fluorescent, IR fluorescent, or visible dye covering the spectrum of 300 to 740 nm, or
(ii) It is labeled with biotin and is detectable with streptavidin bound to a fluorescent, IR fluorescent, or visible dye covering the spectrum of 300 to 740 nm, and/or
(iii) The amount of said at least one ssDNA reference tag oligo and/or of said at least one ssRNA reference tag oligo is 2-fold to 5-fold greater than the mass of the two closest polyA oligos with which it migrates during said electrophoretic separation.

9. The polyA size marker ladder of claim 8, wherein said polyA size marker ladder further comprises:
(i) one or more reference tag oligos selected from the group consisting of at least one ssDNA reference tag oligo that has a number of nucleic acid bases that is an integer “Z-1” selected from the group consisting of: 25, 30, 45, 75, 90, 100, 150, 175, 200, 225, 275, 300, 350, 400, 450, 500, 540, 600, 630, 670, 710, 750, 790, 830, 880, 920, 970, and 1000; or
(ii) one or more ssRNA reference tag oligos that has a number of nucleic acid bases that is an integer “Z-2” selected from the group consisting of: 25, 30, 45, 75, 90, 100, 150, 175, 200, 225, 275, 300, 350, 400, 450, 500, 540, 600, 630, 670, 710, 750, 790, 830, 880, 920, 970, and 1000; or
(iii) comprises one ssDNA oligonucleotide of 100 nucleic acid bases in length that has a mass that is 2-fold to 5-fold greater than the 80-base and 100-base poly(A) oligos in said polyA size marker ladder.

10. A kit or kit-of-parts comprising
a) RNase A, and
b) the poly A size marker ladder of claim 1.

11. (canceled)

12. The kit or kit-of-parts of claim 10, further comprising
c) an RNase A reaction buffer, and/or
d) RNase-free water.

13. The kit or kit-of-parts of claim 12, further comprising an electrophoresis gel.

14. The kit or kit-of-parts of claim 10, further comprising a poly(A) oligo binding and/or staining dye.

15. The kit or kit-of-parts of claim 14, wherein said poly(A) oligo binding and/or staining dye is selected from the group consisting of ethidium bromide, coralyne, berberine, in particular berberine hydrochloride, protoberberine, palmatine, jatrorrhizine, coptisine, berberrubine, harmine, harmaline, harmane, harmalol, tryptoline, amarastelline A, annuloline, serpentine, peperine and oxazole gold, wherein said poly(A) oligo binding and/or staining dye is preferably berberine, in particular berberine hydrochloride.

16-26. (canceled)

27. A method comprising:
a) digesting mRNA comprising a poly(A) tail with RNase A;
b) parallel electrophoresis: i) the RNase A digestion products of step a), and ii) a polyA size marker ladder of claim 1; and
c) comparing the size of the poly(A) oligos of said polyA size marker ladder with the size of the poly(A) tails released from said mRNA in step a).

28. The method of claim 27, wherein in step b) said RNase A digestion products and said polyA size marker ladder are electrophoresed on a electrophoresis gel in different lanes.

29. The method of claim 27, wherein prior step b) the RNase A digestion products of step a) and the polyA size marker ladder are heat-denatured in Stop/Loading Buffer.

30. The method of claim 28, wherein following completion of said electrophoresis in step b), said gel is treated with a dye.

31. The method of claim 30, wherein said dye is selected from SYBR-Gold or Oxazole Gold cyanine dye, and wherein said gel is also treated with berberine hydrochloride.
